(19) United States
(12) Patent Application Publication
(10) Pub. No.: US 2009/0075382 A1
(43) Pub. Date: Mar. 19, 2009

(54) FIBRE-REINFORCED SCAFFOLD

(76) Inventor: Eleftherios Sachlos, Hamilton (CA)

Correspondence Address: SAND & SEBOLT, AEGIS TOWER, SUITE 1100, 4940 MUNSON STREET, NW, CANTON, OH, 44718-3615, US

(21) Appl. No.: 12/282,814

(22) PCT Filed: Mar. 13, 2007
(86) PCT No.: PCT/GB2007/000869

§ 371 Date: Sep. 12, 2008

(30) Foreign Application Priority Data
Mar. 14, 2006 (GB) 0605114.8

(51) Int. Cl. C12N 5/02

(52) U.S. Cl. 435/398

(57) ABSTRACT

The invention provides a fibre-reinforced scaffold for tissue engineering. The scaffold comprises a matrix comprising a biocompatible polymer, the matrix having a, porous structure; and discrete, macroscopic fibres embedded within the matrix, wherein the fibres are oriented such that at least one mechanical property of the scaffold is anisotropic. The invention further relates to fibre-reinforced films and to processes for producing such scaffolds and films.

### FIELD OF THE INVENTION

The invention relates to fibre-reinforced scaffolds and fibre-reinforced films for use in tissue engineering. The invention further relates to processes for producing such scaffolds and films.

### DESCRIPTION OF THE PRIOR ART

Tissue engineering involves the development of biological substitutes that restore, maintain or improve tissue function. This field has the potential of overcoming the limitations of conventional treatments by producing a supply of organ and tissue substitutes biologically tailored to the patient.

Tissue engineering involves growing the relevant cell(s) in the laboratory into the required organ or tissue. However, unaided cells lack the ability to grow in favoured orientations and thus define the anatomical shape of the organ and tissue. Instead, they randomly migrate to form a two dimensional layer of cells. Thus, three dimensional (3D) tissues are required and this is achieved by the use of 3D scaffolds, which act as substrates for cellular attachment. Typically, scaffolds are required to 1) have porosity, generally interconnecting, so as to allow tissue integration and blood vessel colonisation, 2) be made of a biodegradable or bioresorbable material so that tissue can eventually replace the scaffold as it degrades, 3) have appropriate surface chemistry to favour cell attachment, proliferation and differentiation, 4) possess adequate mechanical properties to match the intended implantation site and 5) be easily fabricated into a variety of shapes and sizes. In particular, the pore size of the scaffold has been identified as critical for the successful growth of tissues. For example, an average pore size range of 200 to 400 μm has been shown as optimum for the growth of bone tissue.

Biodegradable and bioresorbable polymers and ceramics have been used as the material to make the scaffolds. The majority of the work has focussed on polymers, however, since ceramic scaffolds have been aimed mostly at bone tissue engineering. The polymers which have been used are synthetic (e.g. polylactic acid and polyglycolic acid, FDA approved polymers used for sutures and orthopaedic fixation screws), or natural (e.g. collagen, an abundant protein present in the connective tissue of mammals which is FDA approved).

Scaffolds that contain fibres are known. WO 98/53768, for example, describes scaffolds containing a matrix of a random copolymer of 75:25 poly(D,L-lactide-co-glycolide), with poly(glycolide) fibers distributed therein. The fibres are of about 15 μm in diameter and about 2.5 mm in length. The scaffolds are made by mixing a suspension of the poly(glycolide) fibres in ethanol with an acetone solution of the matrix copolymer. The matrix copolymer is insoluble in ethanol, so upon mixing, the matrix copolymer precipitates together with the fibres as a composite gel. The gel is separated from the supernatant and foamed in a vacuum oven in order to form the porous, fibre-containing scaffold. For the method to work, the fibres of the scaffold should be insoluble in the solvent used to dissolve the matrix polymer. The fibres should therefore be made of a different material from that of the polymer matrix.

Attempts have been made, during manufacture of fibre-containing scaffolds, to orientate the fibres within the scaffold matrix in one particular direction, or in one particular plane. Indeed, WO 98/53768 teaches that the fibres of a composite fibre-polymer gel may be oriented predominantly in a single direction by hand-rolling the gel into a cylindrical shape. In doing this, the fibres are said to become oriented predominantly in the direction of the length of the cylinder. The gel can then be foamed in a cylindrical mould to produce a porous scaffold which is said to have preferentially orientated fibres. Alternatively, WO 98/53768 teaches that the gel may be flattened and then foamed in a flat mould, in order to orientate the fibres predominantly within a single plane.

The use of fibres within a scaffold matrix could potentially provide the scaffold with advantageous mechanical properties. However, developments in this direction have thus far been hampered by limitations in the methods used to form the scaffolds and position the fibres, and in the nature of the fibres themselves. A further problem is that the fibres of known scaffolds are generally required to be made of a different material from that of the porous matrix in which they are embedded, which requires the use of multiple organic solvents (e.g. acetone, chloroform and methylene chloride). The use of multiple organic solvents increases the risk of one or more solvents remaining as potential carcinogenic/mutagenic or cytotoxic residues within the matrix. Residual organic solvents can compromise the biocompatibility of the porous matrix.

There is therefore a continuing need to address these and other issues in order to prepare fibre-reinforced scaffolds with improved performance characteristics.

### SUMMARY OF THE INVENTION

The present inventors have found that selectively positioning discrete, macroscopic reinforcing fibres within a porous scaffold matrix can give rise to advantageous anisotropic mechanical properties in the resulting scaffold. The anisotropy can be tailored to mimic the mechanical properties of the organ or tissue for which the scaffold is to act as a replacement, and is particularly important for making scaffolds for meniscal, heart valve, blood vessel and tendon regeneration.

Accordingly, in a first aspect the present invention provides a fibre-reinforced scaffold for tissue engineering comprising:

a matrix comprising a biocompatible polymer, the matrix having a porous structure; and

discrete, macroscopic fibres embedded within the matrix, wherein the fibres are oriented such that at least one mechanical property of the scaffold is anisotropic.

The invention further provides a process for producing a fibre-reinforced scaffold according to the invention, the process comprising:

(a) placing said fibres in a mould, orienting the fibres in an arrangement necessary to provide said at least one anisotropic mechanical property and placing in the mould a solution or dispersion of said biocompatible polymer, the mould being a negative of the desired shape of the scaffold;

(b) solidifying the biocompatible polymer having the fibres embedded therein, and removing the mould and the solvent.

Step (a) may involve first placing the fibres in the mould and subsequently adding the solution or dispersion. Alternatively, the solution or dispersion may be added to the mould prior to placing the fibres in the solution or dispersion. The fibres may be oriented in the mould either in the presence or in the absence of the solution or dispersion.

The fibres are accurately positioned within the mould in the orientations which are desired in the final product. Each fibre can be positioned manually in the desired orientation, which allows for a greater degree of control over the anisotropic mechanical properties of the resulting scaffold. Alternatively, the fibre orientation process can be automated by adopting practices from the textile industry. Mechanical machinery designed to manipulate fibres in the dry state can be employed to create numerous fibre patterns. The fibre patterns can then be incorporated into the moulds and filled with matrix solution or dispersion using conventional injection moulding technology.

In this process the fibres may be crosslinked prior to placing the fibres in the mould together with the solution or dispersion of the biocompatible polymer. The fibres may be crosslinked using a chemical treatment, for example with glutaraldehyde or carbodiimide, or using a physical treatment, for example by irradiation with gamma, UV, or microwave radiation or by dehydrothermal treatment. Advantageously, fibres comprising the same biocompatible polymer as that of the matrix may be crosslinked so that they can maintain their integrity during processing. In this way, the crosslinked fibres can be positioned in the solution/dispersion of the matrix polymer without themselves being dispersed or dissolved. Thus, using this technique, the present inventors have been able to design scaffolds in which the porous matrix and the fibres comprise the same polymer, typically collagen. In these scaffolds, the structural integrity of the fibres, as discrete and distinct entities within the bulk porous matrix, is maintained, as are the advantageous anisotropic mechanical properties. The fact that the porous matrix and the fibres comprise the same polymer advantageously removes the necessity to use multiple organic solvents, which could remain as potentially detrimental residues and compromise the biocompatibility of the scaffold.

Thus, in another aspect the present invention provides a fibre-reinforced scaffold for tissue engineering comprising:

a matrix comprising a biocompatible polymer, the matrix having a porous structure; and

discrete fibres embedded within the matrix, wherein the fibres are oriented such that at least one mechanical property of the scaffold is anisotropic, and wherein the fibres comprise the same said biocompatible polymer.

The scaffolds of the invention may comprise a film. Typically, the film is disposed on a surface of the scaffold matrix for providing a smooth surface to the scaffold. Without such a film the surface of the scaffold is relatively rough owing to the porous nature of the scaffold matrix. Thus a film interfaced with a surface of the scaffold matrix advantageously allows for articulation of the scaffold against cartilage or bone in vivo. Interfacing a film with a porous section is particularly beneficial in the fabrication of meniscal scaffolds, where the smooth film surface allows for articulation and the porous component is necessary for the colonisation of meniscal cells. A similar design can be used for heart valve and blood vessel tissue engineering, where the porous component provides the sites for cell colonisation and the smooth film surface reduces turbulence in the blood.

Thus, in another aspect the present invention provides a fibre-reinforced scaffold for tissue engineering comprising:

a matrix comprising a biocompatible polymer, the matrix having a porous structure;

discrete fibres embedded within the matrix; and

a film comprising a biocompatible polymer,

wherein the fibres are oriented such that at least one mechanical property of the scaffold is anisotropic.

Typically, the film is non-porous. Typically, the film is disposed on a surface of the scaffold matrix. However, alternative embodiments are envisaged where the film is included in the interior of the scaffold matrix.

Fibres can be used to reinforce a film in preferential directions. This is accomplished by selectively positioning the fibres in a dispersion of material which is then evaporated to create a membrane.

Accordingly, in another aspect the invention provides a fibre-reinforced film for use in tissue-engineering comprising: a non-porous matrix comprising a biocompatible polymer, the matrix having a thickness of less than 3 mm; and discrete, macroscopic fibres embedded within the matrix, wherein the fibres are oriented such that at least one mechanical property of the film is anisotropic.

Preferably the fibres comprise the same biocompatible polymer as the matrix.

The invention further provides a process for producing a fibre-reinforced film of the invention, the process comprising:
(a) forming a layer of a solution or dispersion comprising the biocompatible polymer, and orienting the fibres in an arrangement necessary to provide said at least one anisotropic mechanical property; and
(b) drying the solution or dispersion to produce said fibre-reinforced film.

Step (a) can involve adding the solution or dispersion to the fibres, or, alternatively, forming said layer of solution or dispersion first and subsequently placing the fibres in the layer. The fibres may be oriented either in the absence or in the presence of the solution or dispersion (i.e. either before or after addition of the solution/dispersion to the fibres).

In step (b), the solution or dispersion is typically air-dried.

The fibre-reinforced scaffolds of the invention having a film disposed on a surface of the scaffold matrix may advantageously be produced in a single mould.

Thus, the invention further provides a process for producing a fibre-reinforced scaffold of the invention comprising a film disposed on a surface of the scaffold matrix, the process comprising:
(a) forming, in a mould, a layer of a solution or dispersion of a first biocompatible polymer, wherein the mould is a negative of the desired shape of the scaffold;
(b) drying the solution or dispersion to produce a film of said first biocompatible polymer;
(c) placing said fibres in a mould, orienting the fibres in an arrangement necessary to provide said at least one anisotropic mechanical property and placing in the mould a solution or dispersion of a second biocompatible polymer;
(d) solidifying the second biocompatible polymer having the fibres embedded therein, and removing the mould and the solvent.

In this process, the first biocompatible polymer is that of the film, and the second biocompatible polymer is that of the scaffold matrix. In step (b), the solution or dispersion is typically air-dried.

The film may be a fibre-reinforced film of the invention. Thus, (a) may further comprise placing fibres in said mould, and orienting the fibres in an arrangement necessary to provide the film with said at least one anisotropic mechanical property. The fibres may be placed in the mould before the solution or dispersion of the first biocompatible polymer is added. Alternatively, the solution or dispersion may be added to the mould prior to placing the fibres therein. The fibres may be oriented in the mould either in the presence or in the absence of the solution or dispersion.

The anisotropic mechanical properties of a fibre-reinforced scaffold can be further enhanced by the use of rope structures embedded in the scaffold matrix. Ropes comprise a plurality of fibres twisted together along their axes. For example, three fibres can be twisted together to form a tri-stranded rope.

Thus, in another aspect, the invention provides a fibre-reinforced scaffold for tissue engineering comprising:

a matrix comprising a biocompatible polymer, the matrix having a porous structure;

discrete fibres embedded within the matrix; and

one or more ropes embedded within the matrix, the ropes comprising a plurality of said fibres entwined together;

wherein the fibres and ropes are oriented such that at least one mechanical property of the scaffold is anisotropic.

In this embodiment, either some or all of the fibres of the reinforced scaffold may be entwined together as ropes.

Anisotropy within scaffolds can be further tailored to mimic the mechanical properties of an organ or tissue by reinforcing the scaffold in more than one particular direction.

Thus, in another aspect, the invention provides a fibre-reinforced scaffold for tissue engineering comprising:

a matrix comprising a biocompatible polymer, the matrix having a porous structure;

a first set of fibres embedded within the matrix, and

a second set of fibres embedded within the matrix,

wherein the fibres of said first set have a common orientation and the fibres of the second set have a common orientation, wherein the orientation of the fibres of the first set is different from the orientation of the fibres of the second set.

The scaffolds and films of the invention may be implanted into humans or animals.

### BRIEF DESCRIPTION OF THE FIGURES

FIG. 1 shows a micrograph of a collagen fibre, as used in the scaffolds of the present invention (160×, 10 kV, 14 mm, GTA—single stranded).

FIG. 2 shows a micrograph of a tri-stranded rope of collagen (90×, 10 kV, 14 mm, GTA—Tri stranded).

FIG. 3 shows a micrograph of a tri-stranded rope of collagen embedded in a film of collagen (100×, 10 kV, 14 mm, Reinforced Membrane—50 degrees).

FIG. 4 shows the stress-strain curves of collagen films both with and without a single reinforcing fibre. Together the curves show the impact of fibre reinforcement on the mechanical properties of collagen films. The y-axis represents stress in units of MPa and the x-axis represents Strain. Curve B is the stress-strain curve of collagen film without a reinforcing fibre and curve A is the stress-strain curve of collagen film with a single reinforcing fibre. The Young's modulus of the film is calculated to be 0.2 GPa whereas the reinforced film has a Young's modulus of 1 GPa. A single reinforcing fibre significantly increased the stiffness of the collagen film.

FIG. 5 is a schematic representation of cross-section through a fibre-reinforced meniscus scaffold according to the present invention. The scaffold has a fibre-reinforced collagen film disposed on its surface. The collagen film has both fibres and tri-stranded ropes embedded therein, so that the top layer of the collagen scaffold is reinforced with collagen fibres and ropes. Labels A to E represent the following structures: A: film; B: porous matrix; C: fibre; D: tri-stranded rope; and E: fibre.

FIG. 6a shows a light micrograph of a collagen scaffold without reinforcing fibres or ropes.

FIG. 6b shows a light micrograph of the top of the meniscus scaffold shown schematically in FIG. 5. The collagen film is relatively smooth and has reinforcing fibres that are dark in colour relative to the white collagen scaffold.

FIG. 7 shows a fibre-reinforced film having fibres (light grey) oriented in parallel within a collagen film (opaque).

FIG. 8 shows a crosshatched fibre-reinforced film. The fibres are multi-directional in orientation, some in the x-axis direction and the others the y-axis direction.

FIG. 9 shows radially-oriented fibres embedded in a collagen film.

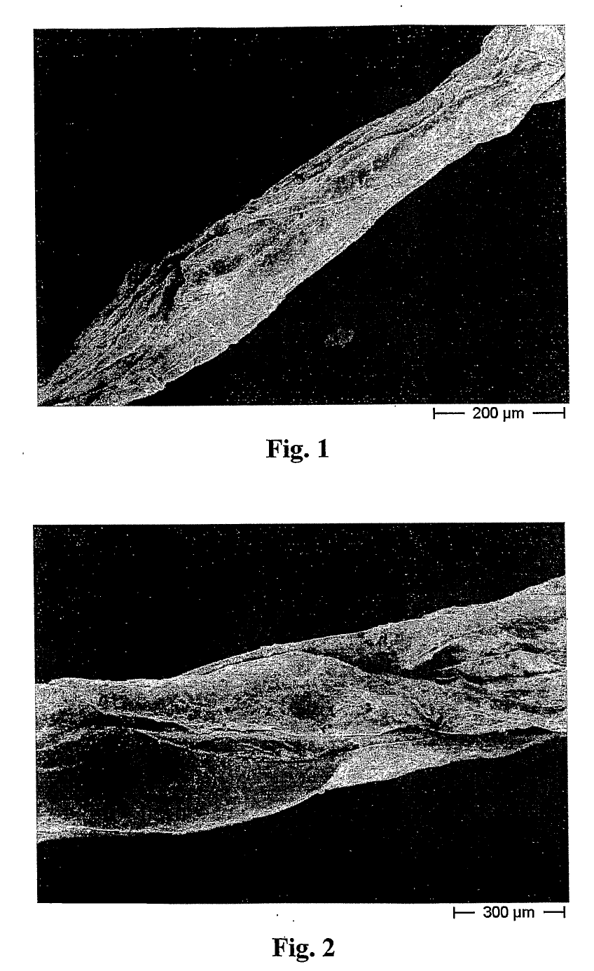

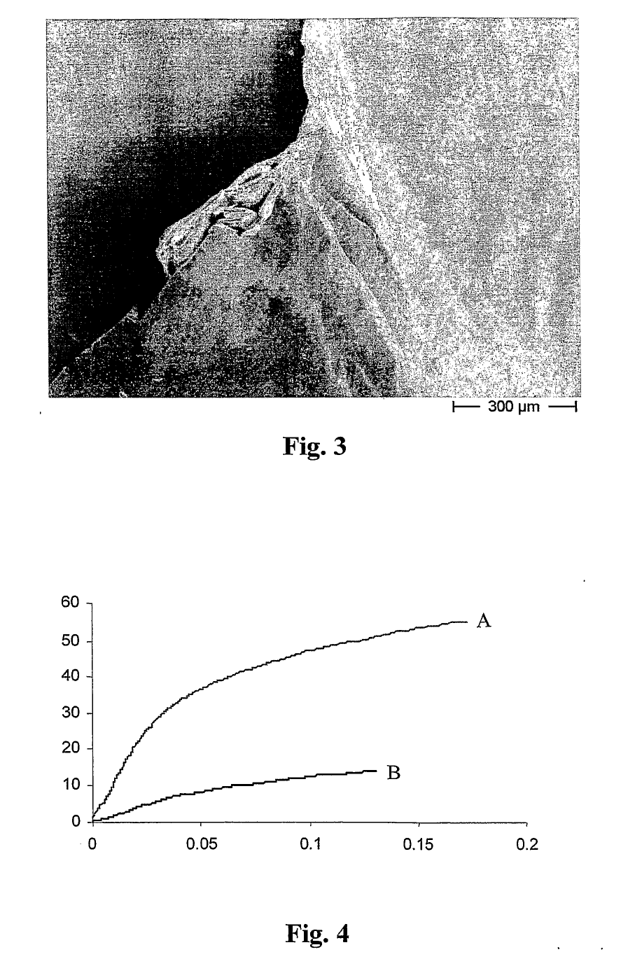

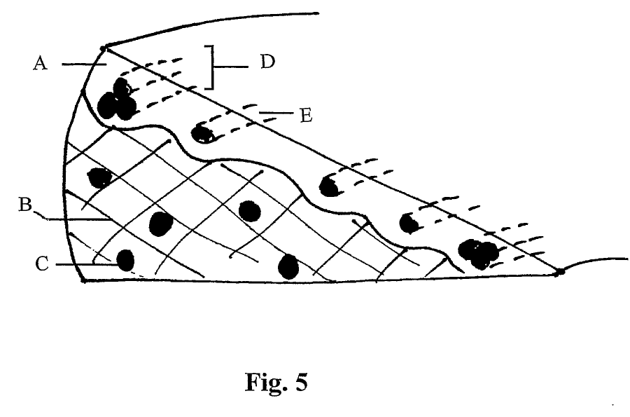

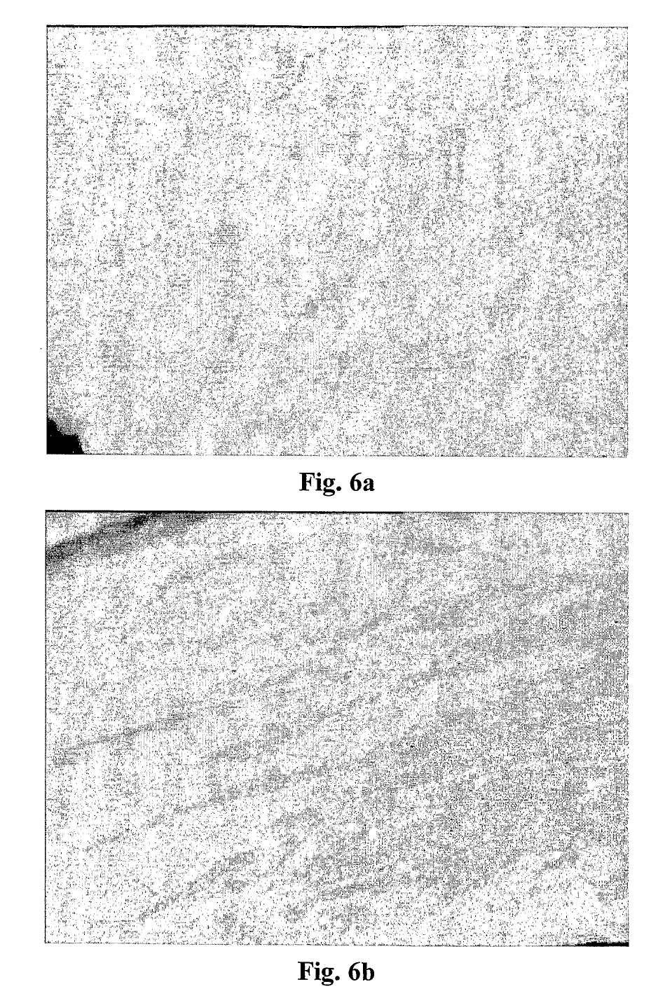

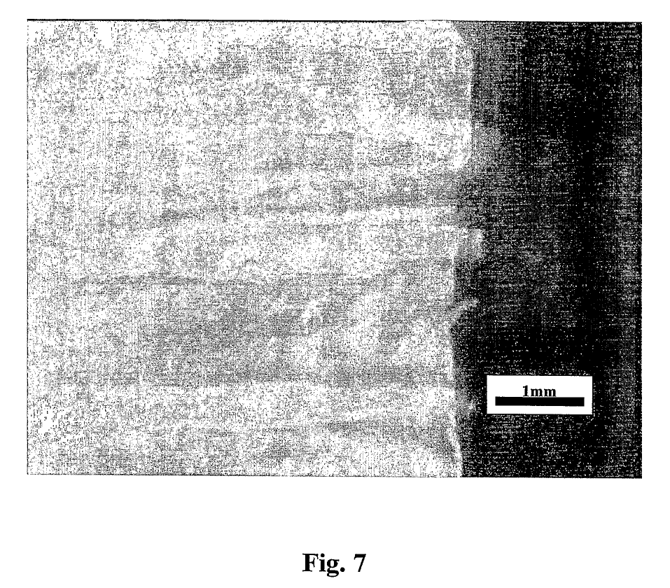

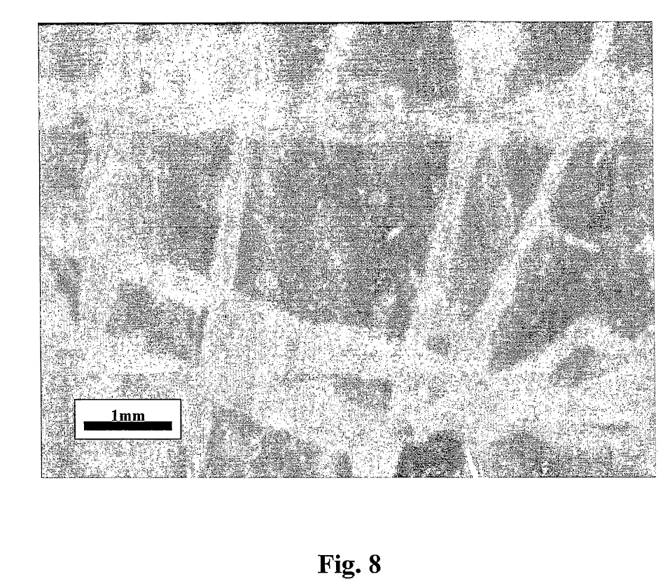

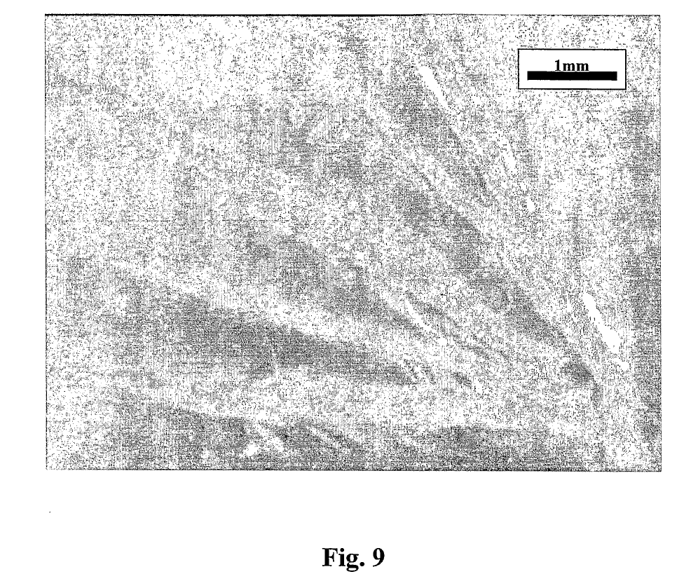

### DETAILED DESCRIPTION OF THE INVENTION

The scaffolds and films of the present invention comprise a matrix, which matrix comprises a biocompatible polymer.

The fibres embedded in the matrix may be polymeric. Alternatively, however, the fibres may comprise, or consist of, a material other than a polymer, such as an inorganic material. The inorganic material may be a metal or a bioceramic, for example. The metal may be titanium. The bioceramic may be a calcium phosphate of hydroxyapatite, tricalcium phosphate, octacalcium phosphate, dicalcium phosphate dehydrate, amorphous calcium phosphate or bioglass or apatite-wollastonite glass ceramic, for example. As a further alternative, the fibres may comprise, or consist of, body tissue, typically non-immunogenic body tissue. The body tissue may be tendon, for example. Typically, the fibres are polymeric. In this context the term “polymeric” means that the fibres comprise one or more polymers. The polymers may be homopolymers or copolymers. Thus, in one embodiment, the fibres comprise a blend of two or more polymers, wherein the polymers may be homopolymers or copolymers.

The polymer or polymers of the scaffolds, films and fibres are biocompatible, and preferably biodegradable or bioresorbable so that tissue can eventually replace the scaffold as it degrades in the body.

Natural polymers are preferred for both the fibres and for the matrix of the scaffold or film. Of these, collagen is particularly preferred, but any other naturally occurring extracellular matrix material can be employed. Suitable naturally occurring polymers, including proteins, polysaccharides, lipids and nucleic acids, include elastin; fibrin; albumen; gelatin; glycoaminoglycans such as hyaluronic acid, chondroitin sulphate, dermatan sulphate, keratan sulphate, heparan sulfate and heparin; and proteoglycans such as aggrecan, versican, neurocan, brevican, decorin, biglycan, fibromodulin, lumican and FACIT collagen; and mixtures thereof. Other natural polymers which are not present in the human body's extracellular matrices but are suitable biomaterials include chitin, chitosan, dextran, amylose, alginate/alginic acid and silk, and mixtures thereof.

Alternatively, the polymer may be a synthetic biodegradable and bioresorbable polymer. Suitable synthetic polymers include polylactic acid; polyglycolic acid and their copolymers; polycaprolactone; polyanhydrides; polyorthoesters; polycarbonates; polyfumarates; poly-L-lysine; poly-L-leucine; poly-L-alanine; poly-L-glutamic acid; poly-α-malic acid; polyphosphazene; polyethyleneglycol-polyester and ethylene oxide-polyester copolymers.

The matrix or fibres may further comprise a second biocompatible material, such as elastin; a glycoaminoglycan; or a bioceramic. The glycoaminoglycan may be selected from hyaluronic acid, chondroitin sulfate, dermatan sulfate, keratan sulphate, heparan sulfate and heparin. The bioceramic may be a calcium phosphate of hydroxyapatite, tricalcium phosphate, octacalcium phosphate, dicalcium phosphate dehydrate, amorphous calcium phosphate or bioglass or apatite-wollastonite glass ceramic. Thus the matrix or the fibres may comprise combinations of the above materials to form composites such as collagen-elastin, collagen-glycoaminoglycan and collagen in combination with bioceramics such as those listed above. A particularly preferred composite for the fibres or matrix is a mixture of collagen and elastin which can, if desired, be crosslinked. Mixtures of any of the synthetic and naturally-occurring polymers listed above may be employed. Alternatively, the matrix or fibres may comprise a copolymer of different monomer units selected from of any of the above polymers.

The skilled person will appreciate that numerous material combinations are possible, for example, collagen fibres can be entrapped in a collagen-elastin membrane or collagen-elastin fibres in a collagen membrane.

In one embodiment, the fibres of the scaffolds and films of the present invention may comprise, or consist of, non-immunogenic tendon. In this context, “non-immunogenic tendon” refers to actual tendons which have been decellurised and processed to be non-immunogenic.

As indicated above, collagen is the preferred material for both the fibres and the matrix used in the scaffolds and films of the present invention. The subsequent description will generally refer to collagen although it will be appreciated that the other biocompatible polymers mentioned above can be used in a similar way. Collagen not only serves as a structural component in many tissues but also as a chemotactic (cell-attracting) agent for several cell types. Therefore collagen exhibits enhanced cellular attachment and provides an environment that resembles more the natural extracellular matrix of the tissue compared to synthetic polymers. The nature of the collagen is not particularly critical. Thus it can be type I collagen as present in bone, skin, tendon, ligaments, cornea and internal organs or type II collagen which is present in cartilage, invertebral disk, notochord and the vitreous humour of the eye. More than 26 genetically distinct collagen types have been discovered to date in varying concentrations in different tissues and more are likely to be discovered in the future. The use of mammalian collagen, from bovine, porcine, equine or ovine sources, is particularly convenient as it is abundant. However, other sources like recombinant human collagen from transgenic animals or genetically engineered bacteria are attractive for this application.

The matrix of the scaffolds of the present invention is porous so as to allow tissue integration and blood vessel colonisation of the scaffold in vivo. By “porous” herein is meant a porosity of from 60% to 99%. Typically, the matrix of the scaffold of the present invention has a porosity of from 80% to 99%, and more typically from 90% to 99%. Typically the average diameter of the pores is from 100 to 400 μm, and more typically from 200 to 300 μm for bone tissue engineering applications. Preferably the matrix pores are interconnected and in association with a 3D network of microchannels, to assist perfusion of the matrix.

The diameter of the pores need not be uniform throughout the matrix, and different regions of the matrix may contain pores of different average diameter. This may be required where different tissue types are required to colonise the different regions of the matrix. For example, a first region of the matrix may comprise pores having an average diameter of 200 μm for colonisation by bone tissue, whereas a second region may comprise pores having an average diameter of 100 μm for colonisation by cartilage. Thus, in one embodiment of the scaffold of the present invention, the matrix of the scaffold has a first porous region and a second porous region, wherein the average pore diameter of the first porous region is different from that of the second porous region.

It is preferred that the fibres embedded within the scaffolds and films of the invention are macroscopic. As used herein “macroscopic” means visible to the naked eye, as opposed to microscopic, which means so small as to be invisible or indistinct without the use of a microscope. The use of larger, macroscopic fibres in a scaffold or film advantageously increases the Young's Modulus of the scaffold or film in the direction of the fibres, which reflects an increased resistance of the material to elongation. Thus the macroscopic fibres can be used to ensure that the scaffolds and films of the invention possess sufficient mechanical properties to match those of the intended implantation site or necessary to the resist the contractile forces exerted by cells. The macroscopic fibres used in the present invention typically have an average diameter of at least 100 μm; more typically the fibres have an average diameter of from 100 μm to 1 mm, and even more typically from 100 μm to 600 μm. However, a plurality of the macroscopic reinforcing fibres can be twisted together along their axes to form a rope. The resulting rope will have a larger diameter than the fibres from which it is made, thus a collection of macroscopic fibres coiled around a common axis can have a diameter ranging from 100 μm to 3 mm, for example. Actual tendons which have be decellurised and processed to be non-immunogenic can also be used as fibres. These fibres may be from 100 μm to 5 mm in diameter. Typically, the length of the macroscopic fibres used in the present invention is greater than 1 cm; more typically the fibres have a length of from 1 cm to 30 cm, and even more typically from 2 cm to 5 cm.

The skilled person will appreciate that collagen is itself a fibrous material. Indeed, collagen proteins are comprised of polypeptide chains that form a triple-helical structure that is 300 nm long and 1.5 nm in diameter. These chains assemble into microfibrils, which typically have a diameter of from 0.3 μm to 1 μm. Thus, the macroscopic fibres used in the scaffold and films of the present invention are of significantly larger size than the microfibrils which would be present in a collagen matrix material, for example.

Typically, both the matrix of the scaffold or film and the reinforcing fibres embedded therein comprise collagen. In that case, the larger size of the collagen reinforcing fibres compared to the microfibrils of the collagen matrix serves to maintain the reinforcing fibres as discrete entities within the matrix. In such scaffolds, the structural integrity of the fibres as discrete and distinct entities can be further enhanced if the fibres are crosslinked. Accordingly, it is preferred that the fibres used in the scaffolds and films of the present invention are crosslinked; that is, chemical bonds or “crosslinks” are preferably present between different polymer chains present within each fibre. In this way, the crosslinks link the polymer molecules within the fibre together, to form a stronger fibre. Where both the fibres and the matrix contain crosslinks, it is preferred that the crosslink density in the fibres is greater than that in the matrix. In this context, “crosslink density” is defined as the mole fraction of monomer units in the polymer that are crosslink points.

Preferably, the fibre-reinforced scaffolds and films of the present invention further comprise one or more ropes embedded within the matrix, in order to enhance the mechanical properties of the scaffold or film. Each rope comprises a plurality of said fibres entwined together. Preferably, each rope contains crosslinks, not only to strengthen the individual fibres of the rope, but also to link the adjacent fibres together in order to strengthen the rope as a whole and decrease the chance of the fibres uncoiling.

Preferably, the fibre-reinforced scaffold is shaped to have the gross shape of the organ or tissue, or of a specific part of the organ or tissue, for which it is to act as a replacement. Thus, the scaffold of the invention may have the gross shape of a meniscus, heart valve, blood vessel or tendon, for example. As the skilled person will appreciate, the dimensions of scaffolds used to make menisci, heart valves, blood vessels and tendons should resemble the anatomical shape and size of the particular organ or tissue in the patient. For example the internal diameter of blood vessels ranges from 4 μm to 30 mm, and menisci and heart valves vary in size greatly between young children and adults. Heart valves are approximately 1.5 mm thick with diameters of 10-30 mm. Menisci are typically C-shaped structures with the inner diameter approximately 3 mm in height and the outer diameter approximately 8 mm in height.

The reinforcing fibres used in the present invention are oriented within the porous scaffold matrix (or within the non-porous film matrix) such that at least one mechanical property of the scaffold (or film) is anisotropic. The mechanical property may be elasticity (Young's Modulus), ultimate tensile stress or tensile strength, yield stress, compressive strength, flexural strength, shear strength, shear modulus, toughness, ductility or impact resistance, for example. The anisotropic mechanical property (or properties) should mimic that (or those) of the organ or tissue for which the scaffold is to act as a replacement. For example, the anisotropic mechanical property may be an increased strength in a particular direction in order to bear a physiological load applied in that direction. For example, a tissue scaffold for a tendon would require an increased strength along the direction between the muscle and the bone that the tendon connects. Thus, the fibres may be oriented in a common direction in order to increase the mechanical strength of the scaffold in that direction. Accordingly, the fibres are preferably oriented in a common direction and said at least one mechanical property is tensile strength. Preferably, the direction corresponds to the direction of physiological load in an organ or tissue for which the scaffold is to act as a replacement. Typically the fibres are oriented parallel to one another, radially or circumferentially, depending on the requirements of the tissue for which the scaffold is to act as a replacement. In the case of a blood vessel tissue scaffold, the fibres may be arranged longitudinally along, helically around or circumferentially around the tubular wall of the scaffold. In another embodiment, the scaffold is generally C-shaped, and the fibres are positioned radially or circumferentially in relation to the curved surface. An example of such a scaffold is a meniscal scaffold, such as the one described in Example 2 herein.

Anisotropy within scaffolds can be further tailored to mimic the mechanical properties of an organ or tissue by reinforcing the scaffold in more than one particular direction. Thus, in one embodiment, the fibre-reinforced scaffold of the invention may comprise a first set of fibres and a second set of fibres, wherein the fibres of said first set have a common orientation and the fibres of the second set have a common orientation, wherein the orientation of the fibres of the first set is different from the orientation of the fibres of the second set.

Typically, the common orientations of the fibres of the first and second sets are independently selected from circumferential, radial, parallel, helical and spiral orientations. Thus, the fibres of the first set may be oriented parallel to one another in a common first direction whilst the fibres of the second set are oriented parallel to one another in a common second direction. In that case the first direction may be substantially perpendicular to the second direction so that the fibres are crosshatched. Alternatively, the fibres of the first set may be oriented circumferentially whilst the fibres of the second set are oriented radially. This latter conformation is advantageous for meniscal scaffolds. Furthermore, as the skilled person will appreciate, different sections within the same scaffold can have different fibre or rope orientations, leading to different sections within the same structure having different mechanical properties.

The fibre-reinforced scaffold of the present invention may further comprise a film. Typically, the film is disposed on a surface of the matrix for providing the scaffold with a smooth surface. The film comprises a biocompatible polymer, which is preferably the same polymer as that of the scaffold on which it is disposed. The polymer of the film may be selected from the list of biocompatible polymers provided herein, including blends and copolymers. Typically the polymer is collagen. Preferably the film is non-porous, so as to provide a smooth surface to the scaffold. Preferably, the film is a fibre-reinforced film according to the present invention. By “non-porous” herein is meant a porosity of less than or equal to 5%. Typically, the films used in the present invention have a porosity of less than 3%, and more typically less than 1%. The thickness of the films used in the present invention is typically less than 3 mm, more typically less than or equal to 1 mm, even more typically from 40 μm to 1 mm, or from 40 μm to 0.5 mm. However, multiple films can be placed on top of each other to create a thicker laminated structure.

A smooth surface may be required in a scaffold to aid articulation of the scaffold in vivo. Thus, in one embodiment the fibre-reinforced scaffold is a meniscal scaffold, having a film disposed on an outer surface of the scaffold matrix. Preferably, the fibres of the fibre-reinforced meniscal scaffold are aligned circumferentially in relation to the curved surface of the C-shaped scaffold.

Alternatively, a smooth surface could be advantageous if placed on the inside surface of a blood vessel scaffold, or on a surface of a heart valve tissue scaffold: in these cases the smooth surface may reduce turbulence in the blood flowing through or adjacent the scaffold. Thus, in one embodiment the fibre-reinforced scaffold is a blood vessel scaffold, having a film disposed on an inner surface of the scaffold matrix. In another embodiment the fibre-reinforced scaffold is a heart valve tissue scaffold, having a film disposed on a surface of the scaffold matrix.

Macroscopic polymeric fibres may be made according to the following process: (a″) hydrating a strand of a membrane of a polymer; (b″) twisting the hydrated strand about its length axis; (c″) forming the twisted strand into a desired shape; and (d″) drying the strand to form the fibre.

As the skilled person will appreciate, steps (a″) to (d″) would take place prior to the steps of the processes of the present invention for producing fibre-reinforced scaffolds and films.

The polymer membrane may be made from any of the suitable biocompatible polymers referred to above, including blends of polymers and copolymers. Typically the polymer is collagen. Membrane formation can be achieved by evaporating the aqueous component of the dispersion. The membrane can range in thickness from several hundred nanometres to several millimetres thick. The membrane is then cut into strands, which can range in width from a few micrometres to several millimetres and be up to several tens of centimetres in length. The strands can be used as is or more preferably coiled to into a fibre. Coiling can be assisted by rehydrating the strand in water before twisting and then allowing to dry again. Multiple fibres can be coiled together to form rope structures, for example three fibres can be twisted together along their axis to create a tri-stranded rope. Again, rehydrating in water can assist in coiling multi-stranded ropes.

The fibres can then be crosslinked using known techniques. For example, collagen can be crosslinked using chemical (i.e. glutaraldehyde or carbodiimide) or physical (gamma, UV, microwave irradiation or dehydrothermal treatment) techniques. Crosslinking aids in preventing the fibres and ropes from uncoiling. In addition, crosslinking helps fibres comprising the same biocompatible polymer as that of the matrix composition maintain their integrity during processing. In this way, the crosslinked fibres can be positioned in the solution/dispersion of the matrix polymer (according to the processes for producing fibre-reinforced scaffolds and films of the present invention) without themselves being dispersed or dissolved. Thus, using this technique, it is possible to produce scaffolds in which the porous matrix and the fibres comprise the same polymer.

The fibres produced according to this process may be employed in the processes of the invention as outlined above.

Typically, in the processes of the invention for producing a fibre-reinforced scaffold or a fibre-reinforced film, a solution or dispersion of the polymer, typically collagen, is cast in the mould. The concentration of the collagen is desirably as high as possible. Usually, a dispersion of the collagen in water is used, typically, with a concentration of from 0.01 to 10% or more, more particularly 0.1 or 0.5 to 5% and especially 0.75 to 2%, weight/volume. The viscosity of the dispersion increases with an increase in the concentration of collagen. Therefore, highly concentrated collagen dispersions possess a high viscosity and are unable to easily flow into small features of the mould. This results in a trade-off between maximising the amount of collagen in the mould and ensuring that the collagen flows into all the fine features of the mould. This complication can be overcome by casting a low viscosity dispersion of collagen into the mould and then inserting a removable absorbent for the liquid such as chromatographic paper into the collagen dispersion. The concentration of collagen in the mould is increased because the paper effectively sucks up the water component of the dispersion. Repeated steps of casting and paper chromatography treatment are usually required to maximise the concentration of collagen in the mould before freezing.

After the collagen composition has been placed in the mould, the fibres, if required, are accurately positioned within the solution/dispersion in the orientations desired in the final product. The fibres can also be positioned within the mould before it is filled with solution/dispersion. Each fibre may be positioned manually in the desired orientation. However, automation of the fibre orientation can also be achieved by adopting practices from the textile industry. Mechanical machinery designed to manipulate fibres in the dry state can be employed to create numerous fibre patterns. The fibre patterns can then be incorporated into the moulds and filled with matrix solution/dispersion using conventional injection moulding technology. Typically each fibre comprises collagen.

Where a non-porous film is desired, the solution or dispersion of the film composition comprising collagen is typically air-dried.

Where a porous scaffold matrix is desired, and a natural polymer such as collagen is used as the biocompatible polymer, the solution or dispersion of the polymer is generally frozen so as to force the collagen into the interstitial spaces. Thus, in the processes of the invention it is preferred that the step of solidifying the biocompatible polymer and removing the mould and solvent comprises: (a′) freezing the solution or dispersion of the biocompatible polymer to produce a solid mixture of the biocompatible polymer and solvent having the fibres embedded therein; and (b′) removing the mould and the solvent from said solid mixture.

However, when a synthetic polymer, such as any of those outlined above, is used as the biocompatible polymer, it may be foamed by incorporating particles, for example salt particles, in the polymer solution or dispersion. Once the polymer is solidified, the scaffold is immersed in water to leach out the salt and leave a porous polymer matrix.

Typically, where a natural polymer such as collagen is used as the biocompatible polymer, the dispersion is first frozen, typically for about 24 hours, and then the mould is removed. The rate at which the dispersion is frozen and the pH have an effect on the resulting pore size. As is known the faster the dispersion is frozen, the smaller the resulting pores will be. Typically the temperature of freezing is from −20° C. for larger pores to −80° C. for the smallest pores, but the size can of course be controlled by adjusting the rate of cooling. Preferably, the temperature of freezing is from −20° C. to −40° C. This technique allows control over the micropores i.e. the pores created by the ice crystals. For other polymers, there is the option of inducing polymerisation of the monomer or crosslinking the polymer after casting into the mould.

Next the mould has to be removed. This must be done in a way which does not adversely affect the polymer. Thus it will be appreciated that it is not possible to use too much heat, as in firing, for this purpose since this would cause the collagen to denature or degrade. Rather, it is preferred to dissolve the mould away using a non-solvent for collagen, generally whilst being kept below 25° C. Collagen is generally stable at a pH of 3 to 10 so that if the mould material is sensitive to weak acid or weak alkali then such solutions can be used to dissolve away the mould. Alternatively, a hydrolysable salt can be used to make the mould and this can be eliminated after the scaffold has formed by the addition of the appropriate hydrolysate.

It is, however, preferred that the mould is removed by the use of a polar solvent since collagen is unaffected by it; in particular, one can use water, a ketone, an ester or an alcohol, especially one with 1 to 6 carbon atoms such as ethanol or 2-propanol or propanone, aryl acetate or an aqueous solution of such a solvent e.g. an aqueous ethanolic solution. Clearly, it is desirable to use a solvent which does not adversely affect human cells in any way in case of any residues while quickly dissolving the mould and for this purpose ethanol is preferred.

The collagen scaffold which remains is generally in the form of a sponge-like material. Immersing a frozen dispersion of collagen in a (polar) non-solvent (typically an alcohol, such as ethanol) dissolves the ice crystals and produces a sponge-like structure similar to that obtained by freeze-drying, the major difference being that the collagen sponge is now suspended in the non-solvent. Furthermore, the non-solvent may be inducing stiffness to the collagen fibrils by dehydrating them. If water is not used, removal of the solvent is crucial. Critical point drying with liquid carbon dioxide can be used for this purpose. The solvent can also be removed by exchanging it with water. In this instance, the collagen sponge does not require critical point drying, and may be used for the subsequent stages of crosslinking and cell culturing, or an intermediate step of freezing the substituted water and freeze-drying the collagen may be incorporated to facilitate crosslinking before cell culturing. It will be appreciated that removal of the solvent by air-drying is generally not appropriate as the surface tension forces created during evaporation result in a collapse of the delicate porous structure one is trying to create.

Another well-established method for creating scaffolds from natural polymers such as collagen involves freeze-drying or lyophilisation. Once the fibres and/or fibre-reinforced films are positioned within the mould, it is filled with solution/dispersion before freezing the solution/dispersion. The ice crystals generated in the scaffold are then removed by freeze-drying the construct, which involves the sublimation of the ice. Freeze-drying is particularly useful if the mould used to make the scaffold can be physically removed without damaging the scaffold.

According to a preferred embodiment, the article is in the non-solvent and subjected to critical point drying. This is a known technique whereby the article is placed in a pressurised container at, for example, 50 bars pressure with liquid carbon dioxide. The alcohol which is the more dense goes to the base of the container and is replaced by the CO₂. Thus it is possible to remove the solvent within the collagen by substituting it with liquid carbon dioxide. If one then increases the temperature from, say, 15-20° C. to e.g. 33-36° C. with a consequent increase in pressure (to 90 bars) the liquid carbon dioxide will gasify and escape. This results in a dry scaffold which is inherently porous and which retains the internal features dictated by the mould. The dry collagen scaffold can then, if desired, be crosslinked to increase the mechanical strength, decrease the antigenicity and decrease the degradation rate of the scaffold. Crosslinking can be accomplished by both physical and chemical techniques. Physical crosslinking can be achieved by dehydrothermal treatment and UV or gamma irradiation. Aldehydes such as glutaraldehyde and formaldehyde, polyepoxy resin, acyl azides, carbodiimides and hexamethylene compounds can be used for chemical crosslinking.

### EXAMPLES

### Example 1

### Fibre- and Rope-Reinforced Collagen Films

Collagen fibres were made by taking a film of collagen and cutting it into 50 mm×2 mm strands. Each strand was then rehydrated with distilled water which made it sticky and twisted around its axis to form a fibre. The fibres were shaped into straight or curved fibres and let to air dry for 24 hours.

Once dry, three fibres again rehydrated and coiled around each other to form a tri-stranded rope of collagen that was then let to dry. The dry ropes and fibres were chemically crosslinked by immersing in a solution of 2.5% w/v glutaraldehyde in ethanol for 1-2 hours and then washed in fresh ethanol for 24 hours before being air-dried.

The ropes and fibres were cut into specific lengths. 10 ml of 1% w/v collagen dispersion was placed in a Petri dish and the fibres and ropes were submerged in the collagen dispersion and selectively positioned to form different patterns, ranging from parallel, radial and crosshatched. The collagen dispersion was then allowed to air dry for 24 hours.

The mechanical properties of these films were tested using a Dynamic Mechanical Analyser (Perkin-Elmer DMA 7). A collagen film reinforced with a single fibre of collagen aligned in the direction of the load was tested in the tensile mode and compared to the control collagen film without reinforcing fibre.

### Example 2

### Fibre- and Rope-Reinforced Scaffold

A mould for a meniscus construct was made using silicone impression material. The floor of the mould was coated with a 2% w/v collagen dispersion. Tri-stranded ropes of collagen described in Example 1 were submerged in the dispersion and aligned circumferentially. Two tri-stranded ropes were placed at the inner and outermost diameter of the mould and three single-stranded fibres were placed between the ropes. The dispersion was allowed to air-dry, creating a film with fibres and ropes embedded within. A 5% w/v aqueous-based collagen dispersion was then used to fill the mould. Several circumferentially-orientated fibres were embedded in this dispersion. The construct was then placed in a freezer at −30° C. This generated a porous structure due to the formation of ice crystals which aggregate the insoluble collagen in the interstitial space and created a porous structure. The pore size of the structure can be controlled by the freezing rate, a fast freezing rate creates small pores whereas a slow freezing rate creates larger pores. The frozen construct is then immersed in ethanol which dissolves the ice crystals. The ethanol is then removed from the scaffold by critical point drying with liquid carbon dioxide. This method exchanges the ethanol for liquid CO₂ (50 atms at 18° C.) which is then heated to 33-36° C. that forces the CO₂ in the supercritical phase which can then be vented out to leave a dry scaffold.

### Example 3

### Fibre-Reinforced Scaffold Having Fibres Oriented in Two Different Directions

A 1% w/v aqueous-based collagen dispersion was then used to fill a 15 mm diameter by 3 mm height polytetrafluoroethylene mould. Three fibres, cut to appropriate lengths, were embedded in the dispersion in the x-axis direction and three fibres, cut to appropriate lengths, in the y-axis direction. The construct was then placed in a freezer at −30° C. This generated a porous structure due to the formation of ice crystals which aggregate the insoluble collagen in the interstitial space and created a porous structure. The pore size of the structure can be controlled by the freezing rate, a fast freezing rate creates small pores whereas a slow freezing rate creates larger pores. Freezing at −196° C. creates approximately 4 μm pores whereas freezing at −30° C. creates 200-300 μm pores. The frozen construct is then immersed in ethanol which dissolves the ice crystals. The ethanol is then removed from the scaffold by critical point drying with liquid carbon dioxide. This method exchanges the ethanol for liquid CO₂ (50 atms at 18° C.) which is then heated to 33-36° C. that forces the CO₂ in the supercritical phase which can then be vented out to leave a dry scaffold.

1. A fibre-reinforced scaffold for tissue engineering comprising:
a matrix comprising a biocompatible polymer, the matrix having a porous structure; and
discrete, macroscopic fibres embedded within the matrix, wherein the fibres are oriented such that at least one mechanical property of the scaffold is anisotropic.

2. The scaffold of claim 1 wherein the fibres have an average diameter of at least 100 μm.

3. The scaffold of claim 1 wherein the average length of the fibres is greater than 1 cm.

4. The scaffold of claim 1 wherein the fibres are polymeric.

5. The scaffold of claim 4 wherein the fibres comprise the same biocompatible polymer as the matrix.

6. The scaffold of claim 1 wherein the biocompatible polymer is biodegradable or bioresorbable.

7. The scaffold of claim 1 wherein the biocompatible polymer is collagen.

8. The scaffold of claim 1 wherein the fibres comprise collagen.

9. The scaffold of claim 1 wherein the fibres comprise non-immunogenic tendon.

10. The scaffold of claim 1 wherein the fibres, the matrix or both the fibres and the matrix further comprise a second biocompatible material.

11. The scaffold of claim 10 wherein said second biocompatible material is elastin, a glycoaminoglycan or a bioceramic.

12. The scaffold of claim 11 wherein the glycoaminoglycan is selected from a group consisting of hyaluronic acid, chondroitin sulfate, dermatan sulfate, keratan sulphate, heparan sulfate and heparin.

13. The scaffold of claim 11 wherein the bioceramic is selected from a group consisting of calcium phosphate of hydroxyapatite, tricalcium phosphate, octacalcium phosphate, dicalcium phosphate dehydrate, amorphous calcium phosphate, bioglass or apatite-wollastonite glass ceramic.

14. The scaffold of claim 4 wherein the fibres contain crosslinks.

15. The scaffold of claim 14 wherein the fibres and the matrix contain crosslinks, and the crosslink density in the fibres is greater than that in the matrix.

16. The scaffold of claim 1 wherein the fibres are oriented in a common direction.

17. The scaffold of claim 16 wherein said direction corresponds to the direction of physiological load in an organ or tissue for which the scaffold is to act as a replacement.

18. The scaffold of claim 1 wherein the fibres are oriented parallel to one another.

19. The scaffold of claim 1 wherein the fibres are oriented radially or circumferentially.

20. The scaffold of claim 1 wherein the scaffold is generally C-shaped.

21. The scaffold of claim 1 wherein the fibres comprise a first set of fibres and a second set of fibres, wherein the fibres of said first set have a common orientation and the fibres of the second set have a common orientation, wherein the orientation of the fibres of the first set is different from the orientation of the fibres of the second set.

22-24. (canceled)

25. The scaffold of claim 1 further comprising one or more ropes embedded within the matrix, each rope comprising a plurality of said fibres entwined together.

26. (canceled)

27. The scaffold of claim 1 wherein the scaffold has the gross shape of an organ or tissue, or of a specific part of an organ or tissue, for which it is to act as a replacement.

28. The scaffold of claim 27 wherein the scaffold has the gross shape of a meniscus, heart valve, blood vessel or tendon.

29. The scaffold of claim 1 wherein the pores of the matrix are interconnected in three dimensions.

30. The scaffold of claim 1 wherein the matrix has a first porous region and a second porous region, wherein the average pore diameter of the first porous region is different from that of the second porous region.

31-54. (canceled)
